(19) United States
(12) Patent Application Publication
(10) Pub. No.: US 2025/0299836 A1
(43) Pub. Date: Sep. 25, 2025

(54) ARTIFICIAL INTELLIGENCE/MACHINE LEARNING-BASED BIOINFORMATICS PLATFORM FOR ENCEPHALOPATHY AND MULTIFACTORIAL EVIDENCE-BASED ANALYSIS METHOD

(71) Applicant: YIH-SHIONG LIN, New Taipei City (TW)

(72) Inventors: YIH-SHIONG LIN, New Taipei City (TW); WEN-PU YEH, New Taipei City (TW); HUI-YU SHEN, Changhua County (TW); VICTOR B. LAWRENCE, Taipei City (TW)

(21) Appl. No.: 19/231,570

(22) Filed: Jun. 9, 2025

Related U.S. Application Data
(63) Continuation-in-part of application No. 17/967,863, filed on Oct. 17, 2022.

(51) Int. Cl. G16H 50/70; G16B 40/00; G16H 10/20; G16H 10/60; G16H 20/00; G16H 50/30; G16H 50/50; G16H 80/00

(52) U.S. Cl. CPC G16H 50/70; G16B 40/00; G16H 10/20; G16H 10/60; G16H 20/00; G16H 50/30; G16H 50/50; G16H 80/00

(57) ABSTRACT

An artificial intelligence/machine learning-based bioinformatics platform for encephalopathy and a multifactorial evidence-based analysis method are provided. The multifactorial evidence-based analysis method includes collecting basic information of a patient through a clinical research device; transmitting the basic information of the patient to a data analysis module for analysis to generate effective medical information; receiving medical interaction information through a collaborative workstation; converting the effective medical information and the medical interaction information into a multifactorial pragmatic clinical trial through the collaborative workstation; comparing the at least one of piece of real mental symptom data with the plurality of pieces of reference mental symptom data of each of the disease models of a model database through a matching device to match the corresponding disease model; and outputting a treatment plan of the corresponding disease model through the matching device.

### CROSS-REFERENCE TO RELATED PATENT APPLICATION

This application is a continuation-in-part application of the U.S. patent application Ser. No. 17/967,863, filed on Oct. 17, 2022, and entitled “ARTIFICIAL INTELLIGENCE/MACHINE LEARNING BASED BIOINFORMATICS PLATFORM FOR ENCEPHALOPATHY AND MEDICAL DECISION IMPROVEMENT METHOD” now pending, the entire disclosures of which are incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a platform, and more particularly to an artificial intelligence/machine learning based bioinformatics platform for encephalopathy and a medical decision improvement method.

### BACKGROUND OF THE DISCLOSURE

In clinical fields such as mental health and encephalopathy, patients often present with high comorbidity and overlapping transdiagnostic symptoms. Traditional diagnosis-specific treatment models are increasingly inadequate for addressing such complexity. Existing systems also lack tools capable of integrating physician-patient interaction data with real-world data (RWD) to generate personalized treatment recommendations. Furthermore, insurance review and healthcare resource allocation require evidence-based AI platforms to enhance the transparency and justification of clinical decisions.

### SUMMARY OF THE DISCLOSURE

In response to the above-referenced technical inadequacies, the present disclosure provides an artificial intelligence/machine learning-based bioinformatics platform for encephalopathy and a multifactorial evidence-based analysis method.

In order to solve the above-mentioned problems, one of the technical aspects adopted by the present disclosure is to provide an artificial intelligence/machine learning based bioinformatics platform for encephalopathy, which includes an evidence-based clinical system and an evidence-based education system. The evidence-based clinical system is configured to obtain real-world data of a patient. The evidence-based clinical system includes a clinical research device capable and a collaborative workstation. The clinical research device capable of collecting and analyzing basic information of the patient to generate effective medical information, and the collaborative workstation is connected to the clinical research device and configured to obtain medical interaction information between a physician and the patient. The collaborative workstation translates a multifactorial pragmatic clinical trial according to the medical interaction information and the effective medical information, and the multifactorial pragmatic clinical trial includes at least one of piece of real mental symptom data. The collaborative workstation includes a model database and a matching device. The model database includes a plurality of disease models, each of the disease models including a plurality of pieces of reference mental symptom data and a treatment plan, and at least one of piece of the reference mental symptom data is different between any two of the disease models. The matching device is connected to the model database; the matching device is configured to compare the at least one of piece of real mental symptom data with the plurality of pieces of reference mental symptom data of each of the disease models. When the plurality of reference mental symptom data of one of the disease models matches the at least one of piece of real mental symptom data, the matching device outputs the treatment plan of the disease model that matches the at least one of piece of real mental symptom data. The real-world data includes the effective medical information and the pragmatic clinical trial. The evidence-based education system is connected to the evidence-based clinical system and configured to selectively modifying the real-world data.

In order to solve the above-mentioned problems, another one of the technical aspects adopted by the present disclosure is to provide a multifactorial evidence-based analysis method. The multifactorial evidence-based analysis method includes: collecting basic information of a patient through a clinical research device; transmitting the basic information of the patient to a data analysis module for analysis to generate effective medical information; receiving medical interaction information through a collaborative workstation; converting the effective medical information and the medical interaction information into a multifactorial pragmatic clinical trial through the collaborative workstation; comparing the at least one of piece of real mental symptom data with the plurality of pieces of reference mental symptom data of each of the disease models of a model database through a matching device to match the corresponding disease model; and outputting a treatment plan of the corresponding disease model through the matching device.

Therefore, in the artificial intelligence/machine learning-based bioinformatics platform for encephalopathy and the multifactorial evidence-based analysis method provided by the present disclosure, by virtue of “the collaborative workstation translates a multifactorial pragmatic clinical trial according to the medical interaction information and the effective medical information, and the multifactorial pragmatic clinical trial includes at least one of piece of real mental symptom data” and “when the plurality of reference mental symptom data of one of the disease models matches the at least one of piece of real mental symptom data, the matching device outputs the treatment plan of the disease model that matches the at least one of piece of real mental symptom data” the element can be used to provide individualized, evidence-based treatment recommendations for patients with encephalopathy presenting complex or overlapping mental health symptoms.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a circuit block diagram of an artificial intelligence/machine learning based bioinformatics platform for encephalopathy according to a first embodiment of the present disclosure;

FIG. 2 is a circuit block diagram of an evidence-based clinical system according to the first embodiment of the present disclosure;

FIG. 3 is a circuit block diagram of an evidence-based education system according to the first embodiment of the present disclosure;

FIG. 4 is a circuit block 1 diagram showing the artificial intelligence/machine learning based bioinformatics platform being connected to a medical database of an official or medical institution according to the first embodiment of the present disclosure;

FIG. 5 is a flowchart of a medical decision improvement method according to a second embodiment of the present disclosure;

FIG. 6 is a flowchart of another configuration of the medical decision improvement method according to the second embodiment of the present disclosure;

FIG. 7 is a flowchart of the medical decision improvement method according to a third embodiment of the present disclosure; and

FIG. 8 is a circuit block diagram of an evidence-based clinical system according to the first embodiment of the present disclosure;

FIG. 9 is a flowchart of a multifactorial evidence-based analysis method according to a fourth embodiment of the present disclosure; and

FIG. 10 is a flowchart of the multifactorial evidence-based analysis method according to a fourth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

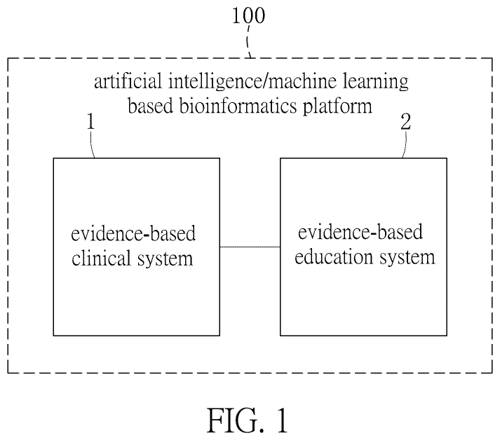

Referring to FIG. 1 to FIG. 4, a first embodiment of the present disclosure provides an artificial intelligence/machine learning (AI/ML) based bioinformatics platform 100 for encephalopathy. In practice, such an AI/ML based bioinformatics platform can also be referred to as a diagnostic formulation developing platform. The AI/ML based bioinformatics platform 100 is configured to integrate a patient's long-term condition, current medical regulations, and an interaction between a physician and the patient, so as to modify (or adjust) a medical decision made by the physician by means of artificial intelligence. In this way, the legality and correctness of medical behavior of the physician toward the patient can be ensured. In other words, the bioinformatics platform 100 in the present embodiment is configured to achieve the aforementioned effects through ICT-Bio translation and integration.

The following description describes the structure and connection relationship of each component of the bioinformatics platform 100.

Referring to FIG. 1, the bioinformatics platform 100 includes an evidence-based clinical system 1 and an evidence-based education system 2 that is connected to the evidence-based clinical system 1. The evidence-based clinical system 1 may be referred to as an evidence-based practical (EBP) tool, and is used to collect real-world data (RWD). The evidence-based education system 2 can also be referred to as an evidence-based educational instrument, and is used to generate real-world evidence (RWE) for confirming (or modifying) the real-world data.

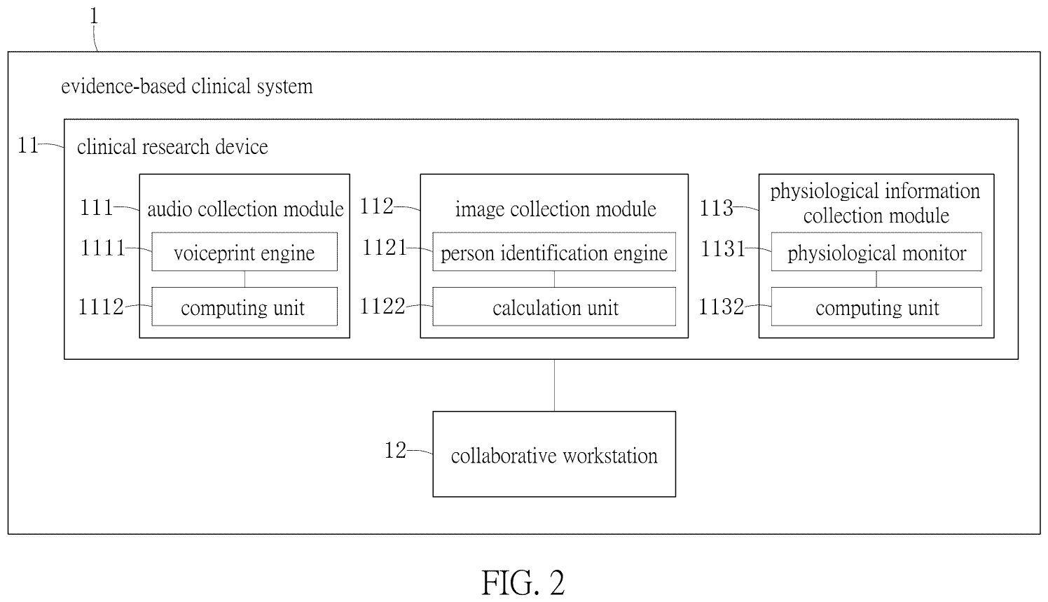

Specifically, as shown in FIG. 1 and FIG. 2, the evidence-based clinical system 1 includes a clinical research device 11 and a collaborative workstation 12 that is connected to the clinical research device 11. The clinical research device 11 is configured to collect basic information of the patient and their surroundings, and the basic information may include at least one of sound data, image data, and physiological data of the patient. When the clinical research device 11 collects the basic information, the clinical research device 11 is configured to analyze the basic information for generation of effective medical information. Here, the effective medical information refers to “information that can be indicated as medical behavior”.

In the present embodiment, the basic information is described as including the audio data, the image data and the physiological data, but the present disclosure is not limited thereto. In other words, the clinical investigation device 11 in the present embodiment includes an audio collection module 111, an image collection module 112, and a physiological information collection module 113.

Specifically, the audio collection module 111 is configured to collect the sound data and analyze the sound data to generate the effective medical information of the patient. In a practical application, the audio collection module 111 includes a voiceprint engine 1111 and a computing unit 1112. The voiceprint engine 1111 can use a natural language processing (NLP) technology to identify a voiceprint of the patient, and provide the same to the computing unit 1112 for analysis, so as to generate the effective medical information.

The sound data can be exemplified to include a first chat content, a second chat content, and a third chat content. The first chat content is of a dialogue between two family members of the patient, the second chat content is of a complaint made by the patient to their pet, and the third chat content is of the patient saying good night to their father. The voiceprint engine 1111 can recognize the voiceprint of the patient, and further transmit the second chat content and the third chat content to the computing unit 1112 for analysis. When the computing unit 1112 finds that the second chat content has symptoms of emotional distress, the computing unit 1112 will define the second chat content as the effective medical information. Naturally, the effective medical information is not limited to language. Depending on different diseases, the effective medical information may be coughing sounds, wheezing sounds, etc.

Furthermore, the image collection module 112 can be a 3D image processing lens, and can be used to collect the image data. In a practical application, the image collection module 112 includes a person identification engine 1121 and a calculation unit 1122. The person identification engine 1121 can identify the patient, and the computing unit 1122 can analyze the image data to generate the effective medical information of the patient.

For example, the image data is assumed to include a first image content, a second image content, and a third image content. The first image content shows the patient pounding on their heart, the second image content shows the family member of the patient stroking the pet's back and the patient coughing beside the family member, and the third image content shows the pet playing alone at home. The person identification engine 1121 can identify facial features and a body shape of the patient, such that the first image content and the second image content are selected for the computing unit 1122 to analyze. Further, only body images corresponding to a disease behavior of the patient are captured by the computing unit 1122 for being used as the effective medical information. In other words, the second image content will be further processed, such that only the image of the patient coughing is left. The first image content does not need to be processed.

In addition, the physiological information collection module 113 can be used to collect and analyze the physiological data of the patient, so as to generate the effective medical information of the patient. In a practical application, the physiological information collection module 113 may include a physiological monitor 1131 (e.g., a smart wearable bracelet and a heart rate monitor) and a computing unit 1132. The physiological monitor 1131 can monitor the physiological data of the patient (e.g., blood pressure, heartbeat, electrocardiogram, body temperature, daily steps, and brain waves) and provide the same to the computing unit 1132 for analysis, so as to generate the effective medical information.

In one example, supposing that the physiological monitor 1131 measures a heartbeat value of the patient at the 49th second to be 60 beats/per minute, a heartbeat value of the patient at the 50th second to be 130 beats/per minute, and a heartbeat value of the patient at the 51th second to be 62 beats/per minute, the computing unit 1132 can determine that the heartbeat value at the 50th second is caused by an abnormality of the device and is to be further excluded (i.e., the heartbeat value at the 50th second is not suitable as data of the effective medical information). Accordingly, the physiological data can be ensured to be correct and can also be used as the effective medical information.

In another example, supposing that the physiological monitor 1131 measures an average diastolic blood pressure of the patient in a first time period to be 75 mmHg, an average diastolic blood pressure in a second time period to be 85 mmHg, and an average diastolic blood pressure in a third time period to be 83 mmHg, the computing unit 1132 determines that the average diastolic blood pressures in the second time period and the third time period are the effective medical information. It should be noted that a diastolic blood pressure of a person is normally less than 80 mmHg.

In addition, the computing units of the audio collection module 111, the image collection module 112, and the physiological information collection module 113 can be integrated into the same chip according to requirements, but details thereof will not be specially described herein.

It should be emphasized that the clinical research device 11 only transmits the effective medical information. That is, data that is not used for the medical behavior will not be transmitted, so as to achieve the personal information protection effect of zero trust. Naturally, the clinical research device 11 in practice is kept connected to the Internet, so as to upload the effective medical information. In addition, when the clinical research device 11 fails to be connected to the Internet, the clinical research device 11 can continue obtaining the effective medical information, so that the effective medical information can be uploaded when the clinical research device 11 is connected to the Internet.

It should be noted that the clinical research device 11 can cooperate with an artificial intelligence technology (e.g., an artificial intelligence module), so as to further guide the patient to communicate. In this way, the basic information that is more conducive to generating the effective medical information can be obtained. That is to say, the clinical research device 11 is a verbal communication mechanism that is capable of active inquiry, passive listening, and interactive communication.

In a practical application, the clinical research device 11 can also be referred to as a biological automated collection/detector for expeditionary reconnaissance (BioACER) edge device. The clinical research device 11 can include an interactive neuro-linguistic programming (NLP) voiceprint engine that has a high directivity, a three-dimensional image processing lens, a variety of psychological/emotional response mechanism software programs, and an artificial intelligence of things (AIOT) terminal device that includes a variety of biosensor elements and switch elements. Accordingly, the clinical research device 11 is suitable for being used as a home-type physiological monitoring instrument.

From another perspective, the clinical research device 11 adopts a machine learning structure. That is to say, the clinical research device 11 can use convolutional neural networks (CNN), recurrent neural networks (RNN), and long short-term memory (LSTM) models to achieve training and recognition of images and voices.

As shown in FIG. 2, the collaborative workstation 12 is used to obtain medical interaction information between the physician and the patient, and the collaborative workstation 12 translates a pragmatic clinical trial (PCT) according to the medical interaction information and the effective medical information. The medical interaction information may refer to information that includes a consultation content between the physician and the patient, the physiological data that is obtained by the physician examining the patient at the time, or a judgment of the physician. The pragmatic clinical trial may refer to a final medical action performed on the patient. The pragmatic clinical trial is used, for example, in setting up a pharmacovigilance. The pharmacovigilance refers to a software, an interface, or an apparatus that performs real-time monitoring of the physician's behavior (e.g., prescribing medicines to the patient and the medication basis for said prescription). The effective medical information and the pragmatic clinical trial can be defined as the real-world data. That is, the real-world data includes the effective medical information and the pragmatic clinical trial.

The collaborative workstation 12 also translates a multifactorial pragmatic clinical trial (MPCT) based on the medical interaction information and the effective medical information. The multifactorial pragmatic clinical trial includes at least one of piece of real mental symptom data, and the multifactorial pragmatic clinical trial refers to an artificial intelligence framework based on the integration of diversified clinical data and cross-diagnostic models, which is used to simulate real-world clinical scenarios and serves as a practical platform to support mental health decision-making, insurance claim denial reviews, and personalized interventions.

More specifically, the multifactorial pragmatic clinical trial is an evidence-based analytical framework that integrates cross-diagnostic treatment characteristics with actual clinical behavioral patterns. It is designed to efficiently and accurately capture multimodal data features involved in clinical environments. By introducing a pragmatic trial approach, the system enhances the effectiveness evaluation and accuracy of mental health interventions in real-world settings.

In addition to providing precise inference for a single diagnostic condition, the multifactorial pragmatic clinical trial is particularly suitable for addressing comorbidities and cross-diagnostic mental and behavioral characteristics. The multifactorial pragmatic clinical trial incorporates artificial intelligence-assisted systems, such as empathetic conversational interfaces, to enable automated data collection, abnormal behavior detection, personalized treatment recommendations, and insurance claim denial review. By linking to continuously updated clinical evidence, the multifactorial pragmatic clinical trial can automatically generate evidence-based documentation with medical legitimacy and regulatory compliance, thereby supporting applications such as insurance appeal, clinical decision support, and treatment optimization for patients.

In a practical application, the collaborative workstation 12 may be, for example, a computer. The collaborative workstation 12 is configured to obtain the medical interaction information between the physician and the patient through the computer, and to translate the medical interaction information into the multifactorial pragmatic clinical trial.

Furthermore, the collaborative workstation 12 may be referred to as an event learning management & surveillance (ELMS) inferencing edge server, and is responsible for “health information management and control, a biometric collection, and patient medical services and interactions at various stages” of the clinical research device 11. In addition, the collaborative workstation 12 can also be switched and transferred to health informatics of the BioACER edge device.

Specifically, the collaborative workstation 12 includes a model database 121, a matching device 122 connected to the model database 121, an emotion recognition module 123 connected to the model database 121, a risk alert device 124 connected to the model database 121, a history tracking device 125 connected to the model database 121. The model database 121 includes a plurality of disease models, each of the disease models includes a plurality of pieces of reference mental symptom data and a treatment plan, and at least one of piece of the reference mental symptom data is different between any two of the disease models.

In a practical application, the model database 121 is composed of a central processing unit 1211, a solid-state drive 1212 connected to the central processing unit, and a router 1213 connected to the central processing unit 1211. The central processing unit 1211 is responsible for receiving the plurality of disease models and storing them in the solid-state drive 1212, such that the matching device 122 can access and quickly load the plurality of disease models stored in the model database 121 at any time through the router 1213.

The matching device 122 is configured to compare the at least one of piece of real mental symptom data with a plurality of pieces of the reference mental symptom data of each of the disease models. When the plurality of pieces of the reference mental symptom data of one of the disease models matches the at least one of piece real mental symptom data, the matching device 122 outputs the treatment plan of the disease model that matches the at least one of piece of real mental symptom data.

In a practical application, the matching device 122 is composed of a central processing unit 1221 and an artificial intelligence computing module 1222. The central processing unit 1221 is used to receive the real mental symptom data and transmit the real mental symptom data to the artificial intelligence computing module 1222 for a matching process. The artificial intelligence computing module 1222 includes at least one neural processing unit 12221 and a graphics processing unit 12222, which are configured to perform the matching computation of the reference mental symptom data of the disease models and to execute machine learning inference, thereby outputting the treatment plan of the disease model that matches the at least one of piece of real mental symptom data.

Furthermore, the evidence-based clinical system 1 is configured to obtain multi-gene testing data of the patient, and the matching device 122 is configured to analyze the multi-gene testing data to adjust the treatment plan. Specifically, when the matching device 122 receives the multi-gene testing data, the matching device 122 adjusts the treatment plan based on the multi-gene testing data in order to improve the accuracy of the treatment plan.

The emotion recognition module 123 is configured to identify an emotional state of the patient based on the basic information, the medical interaction information, and the real mental symptom data. In a practical application, the emotion recognition module 123 includes a central processing unit 1231, an emotion recognition component 1232, and a data transmission unit 1233.

In a practical application, the central processing unit is configured to receive the real mental symptom data and transmit the real mental symptom data to the emotion recognition component for emotion analysis. The emotion recognition component includes at least one neural processing unit 12321 and one graphics processing unit 12322, which are configured to extract and analyze emotional features based on the basic information, the medical interaction information, and the real mental symptom data, and to infer the emotional state of the patient (e.g., anxiety, depression, anger, or calmness). The data transmission unit is a router device configured to store the emotional state of patient and transmit the emotional state to the risk alert device 124.

The risk alert device 124 is configured to generate a personal emotional index based on the effective medical information and the emotional state. The personal emotional index includes a plurality of emotional values of the patient. When a sum of the emotional values exceeds a threshold, the risk alert device 124 issues a warning notification.

In a practical application, the risk alert device 124 is composed of a central processor unit 1241, a memory unit 1242, and a communication module 1243. The central processor unit 1241 is configured to execute a computation logic of the personal emotional index by the effective medical information and the emotional state to generate a plurality of the emotional values, and determine whether the sum of the emotional values exceeds the threshold. The memory unit 1242 is configured to store the threshold setting, historical emotional data, and alert records. The communication module 1243 is configured to trigger a warning condition when the sum of the plurality of the emotional values exceeds the threshold, and to transmit the warning notification to medical personnel or a care system, so that the medical personnel or care system can promptly intervene and provide necessary assistance.

Furthermore, the sum of the plurality of the emotional values calculated by the risk alert device 124 can be transmitted to the clinical research device 11, so that the clinical research device 11 can adjust the interaction and communication strategies for the patient according to the emotional values of the patient.

For example, when the risk alert device 124 detects that one of the emotional values of the patient (e.g., anxiety) exceeds a response threshold, the risk alert device 124 transmits a notification to the clinical research device 11, and the clinical research device 11 can adjust the interaction process with the patient based on the interaction content that triggered the emotional response.

The history tracking device 125 can record and trace back the real-world data, the basic information, the effective medical information, the medical interaction information, the multifactorial pragmatic clinical trial, the real mental symptom data, and the treatment plan related to the patient at any time.

In a practical application, the history tracking device 125 is implemented as a network-attached storage cloud server, which is constructed from a central processing unit 1251, a data processing unit 1252, a solid-state drive 1253, and a blockchain Input/Output (I/O) management module 1254. Within the history tracking device 125, the central processing unit 1251 is responsible for overall system logic control and scheduling of data access instructions. The data processing unit 1252 performs high-speed parallel processing and integration of large volumes of patient-related medical data (e.g., the medical interaction information, the multifactorial pragmatic clinical trial, and the treatment plan). The solid-state drive 1253 provides high-speed storage to support real-time access and historical data retrieval. The blockchain input/output (I/O) management module 1254 can ensure the integrity and immutability of all records and provide a reliable data traceability mechanism, thereby enhancing security and credibility of the system in medical decision-making and regulatory compliance.

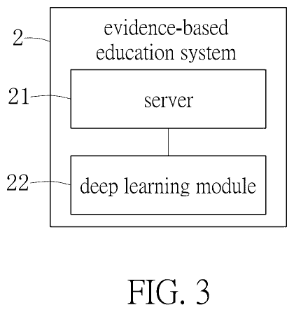

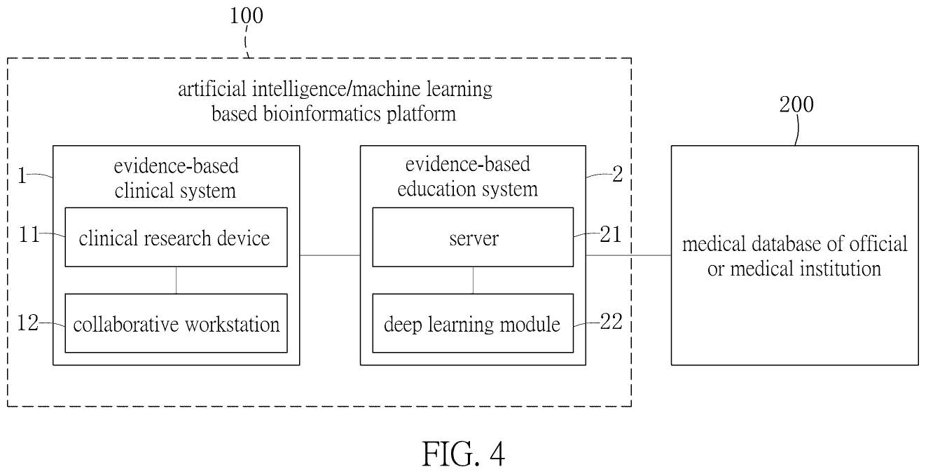

Referring to FIG. 3 and FIG. 4, the evidence-based education system 2 includes a server 21 and a deep learning module 22 that is electrically coupled to the server 21. The server 21 is configured to connect to a medical database of an official or medical institution 200 (e.g., a database of a central health insurance agency), so as to provide legal medical means information for the deep learning module 22. The legal medical means information may include medical history data of the patient (e.g., medical records) and relevant medical regulation data (e.g., drug application regulations and physician laws).

The deep learning module 22 establishes the real-world evidence according to the legal medical means information, the effective medical information, and the medical interaction information. The real-world evidence is configured to selectively modify the real-world data.

For example, as shown in FIG. 1 to FIG. 3, when the physician prescribes (or inputs) a therapeutic drug through the collaborative workstation 12 according to the effective medical information and the medical interaction information, the deep learning module 22 verifies the legality and correctness of the effective medical information and the pragmatic clinical trial (i.e., the real-world data) by using the real-world evidence. When the deep learning module 22 determines that the real-world data is not legal and correct, the deep learning module 22 will modify the real-world data in real time, so as to adjust or reject the medical behavior of the physician. That is to say, the principles generated from the effective medical information by the clinical research device 11 and the medication authorization issued by the collaborative workstation 12 for the physician will be modified in real time.

It should be noted that, since how the deep learning module 22 learns, compares, and analyzes based on multiple pieces of data to achieve verification (or modification) is known to those skilled in the art and is not the focus of the present disclosure, details thereof will be omitted herein.

It can be observed from the above description that the AI/ML based bioinformatics platform 100 of the present disclosure can achieve the function of continuous learning and continuous self-update through the deep learning module 22, so as to further (autonomously) modify the real-world data (or a medical diagnosis process). That is to say, the AI/ML based bioinformatics platform 100 is a platform architecture of an intelligent system (medical robot) for revising the medical diagnosis process.

In addition, in order to avoid inappropriate correction in the deep learning module 22, the data of the deep learning module 22 can be transmitted to a supervisory authority (e.g., the central health insurance agency) for third-party supervision or be sent to a medical hospital (e.g., the collaborative workstation) for recording. In this way, information transparency can be achieved, and artificial intelligence can be prevented from going out of control.

Moreover, since the AI/ML based bioinformatics platform 100 is connected to the medical database of official or medical institution 200 through the server 21, the AI/ML based bioinformatics platform 100 can select from a ranked list of the real-world evidences by use of randomized controlled trials (RCTs), so as to produce disease prediction information.

In conclusion, the AI/ML based bioinformatics platform 100 is established under the framework of MPCT to design an artificial intelligence agent platform that integrates multiple opinions. Based on the MPCT framework, after receiving the basic information and the medical interaction information related to the patient, the AI/ML based bioinformatics platform 100 analyzes the patient's condition and needs, thereby triggering the corresponding functional modules and sending notifications to the relevant medical personnel. The AI/ML based bioinformatics platform 100 can provide the following functions: providing psychosocial support, emotional support, cognitive behavioral therapy, and psychological stress exploration for the patient; identifying the real mental symptom of the patient through the multifactorial pragmatic clinical trial of the patient and offering the corresponding diagnosis and treatment plan; recording the effective medical information of the patient, the medical interaction information of the patient, and the emotional state of the patient, and conducting real-time monitoring and reporting; and performing real-time monitoring of the medical behavior of the physician toward the patient (e.g., prescribed drugs, medication rationale, etc.). Accordingly, under the framework of the multifactorial pragmatic clinical trial, the AI/ML based bioinformatics platform 100 enables flexible, real-time, and personalized integration of diverse perspectives within real-world mental health diagnostic processes.

### Second Embodiment

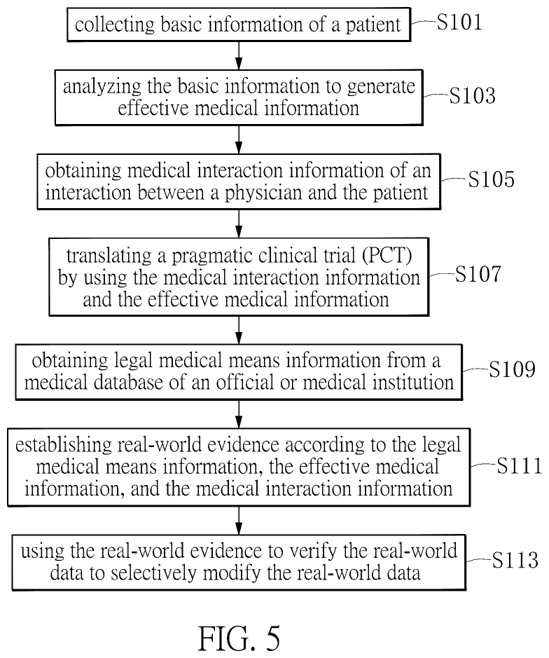

Referring to FIG. 5, a second embodiment of the present disclosure provides a medical decision improvement method, and the medical decision improvement method is applied to a bioinformatics platform (e.g., the AI/ML based bioinformatics platform of the first embodiment). As such, reference is also made to FIG. 1 to FIG. 4. The medical decision improvement method includes steps S101 to S113. It should be noted that any one of the above-mentioned steps can be omitted or adjusted according to practical requirements.

The step S101 is implemented by collecting basic information of a patient. The basic information refers to information about the patient himself/herself or a surrounding environment, and includes at least one of sound data, image data, and physiological data.

The step S103 is implemented by analyzing the basic information to generate effective medical information. The effective medical information refers to information that can be used to indicate medical behavior, such as coughing or mumbling. In a practical application, the basic information can be analyzed by a device with an analysis function (e.g., a computing module, a deep learning module, and a classifier) to generate the effective medical information.

The step S105 is implemented by obtaining medical interaction information of an interaction between a physician and the patient. The medical interaction information refers to any medical behavior between the physician and the patient, such as a consultation between the physician and the patient or issuance of a prescription by the physician based on a diagnosis result.

The step S107 is implemented by translating a pragmatic clinical trial (PCT) by using the medical interaction information and the effective medical information. The pragmatic clinical trial and the effective medical information are defined as real-world data. It should be noted that the pragmatic clinical trial refers to a final medical action performed on the patient.

The step S109 is implemented by obtaining legal medical means information from a medical database of an official or medical institution. The medical database of the official or medical institution may, for example, be a database of a central health insurance agency, and the legal medical means information may include at least one of medical history data of the patient (e.g., medical records) and relevant medical regulation data (e.g., drug application regulations and physician laws).

The step S111 is implemented by establishing real-world evidence according to the legal medical means information, the effective medical information, and the medical interaction information. The real-world evidence is used to verify the legality and correctness of a medical decision.

The step S113 is implemented by using the real-world evidence to verify the real-world data to selectively modify the real-world data. Specifically, when the real-world data is verified by the real-world evidence (that is, the real-world data is legal and correct), the real-world data is executable. Conversely, when the real-world data is not verified by the real-world evidence (that is, the real-world data is not legal and correct), the real-world data can be modified for legality and correctness, or execution of the real-world data can be refused.

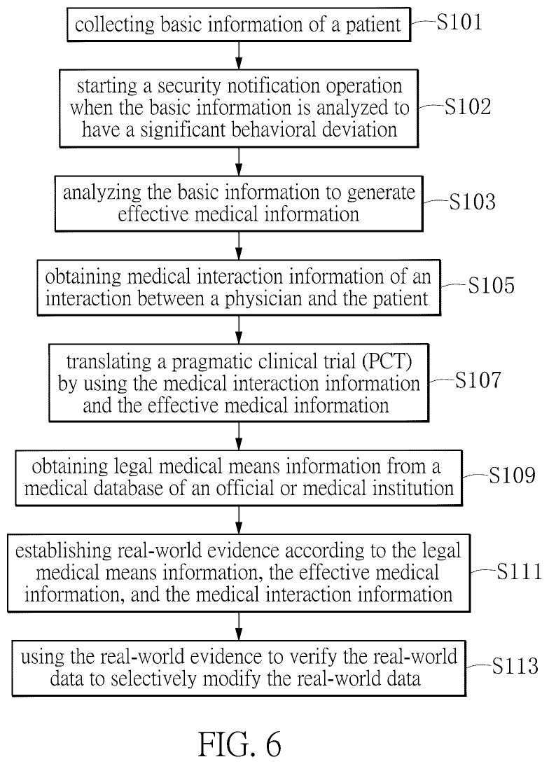

In addition, as shown in FIG. 6, the medical decision improvement method in another configuration can further perform a deviation analysis on the patient based on the basic information. Specifically, the medical decision improvement method further includes step S102 after the step S101. The step S102 is implemented by starting a security notification operation when the basic information is analyzed to have a significant behavioral deviation. The significant behavioral deviation means that the patient is engaging in or intending to engage in an unlawful act (e.g., hurting other people), and the security notification operation is to notify and provide information (e.g., sound or video) corresponding to the significant behavioral deviation to a relevant regulatory agency (e.g., hospitals and law enforcement units).

### Third Embodiment

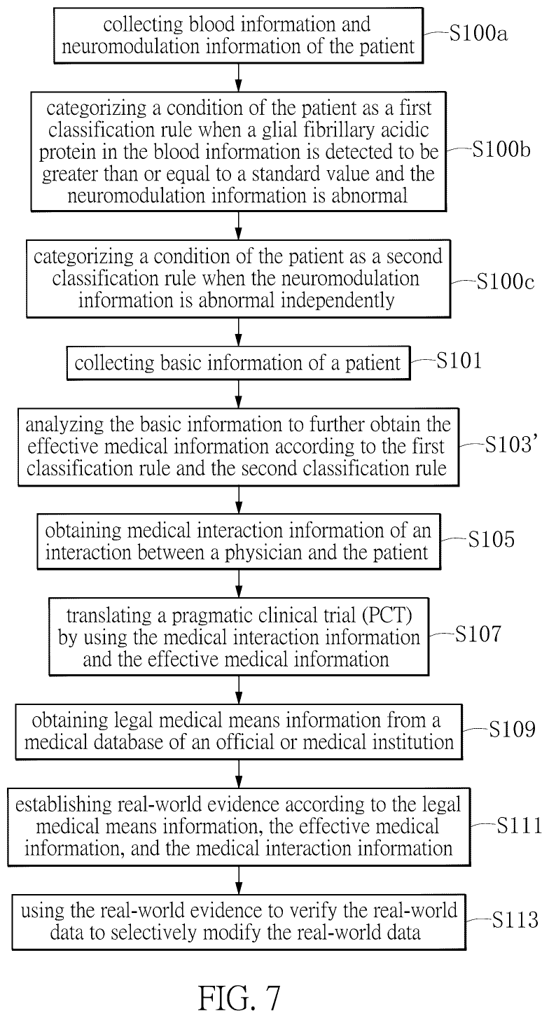

Referring to FIG. 7, a third embodiment of the present disclosure provides a medical decision improvement method. The present embodiment is similar to the second embodiment, and the similarities therebetween will not be repeated herein. The main difference between the present embodiment and the second embodiment is that the medical decision improvement method of the present embodiment further includes steps S100a, S100b, and S100c before the step S101.

The step S100a is implemented by collecting blood information and neuromodulation information of the patient. Specifically, the blood information and the neuromodulation information of the patient can be obtained through a physiological acquisition device (e.g., a blood tester) and a behavior monitoring device (e.g., the clinical research device of the first embodiment, or a camera). The blood information is composition of blood components of the patient, and the neuromodulation information is a long-term dynamic behavior of the patient with respect to neuromodulation.

The step S100b is implemented by categorizing a condition of the patient as a first classification rule when a glial fibrillary acidic protein in the blood information is detected to be greater than or equal to a standard value and the neuromodulation information is abnormal. The standard value is an amount of the glial fibrillary acidic protein in the blood that is sufficient to be judged as brain damage (e.g., leakage of a brain tissue fluid), and detection of the glial fibrillary acidic protein from the blood information can be achieved by a biosensor of chemiluomescence. The abnormal neuromodulation information refers to an abnormal dynamic behavior of the patient (e.g., self-harm).

The step S100c is implemented by categorizing a condition of the patient as a second classification rule when the neuromodulation information is abnormal independently.

After the step S101 (i.e., the basic information of the patient is collected), the medical decision improvement method of the present embodiment proceeds to step S103′. The step S103′ is implemented by analyzing the basic information to further obtain the effective medical information according to the first classification rule and the second classification rule. Then, after the step S103′ is executed, the steps S105 to S113 are executed.

Accordingly, the steps S100b and S100c can be used to determine whether the patient is classified into the first classification rule or the second classification rule. This helps the effective medical information be classified according to causes of emotional distress, e.g., an emotional distress caused by a physical injury and a mental illness (i.e., the first classification rule) or an emotional distress caused by the mental illness (i.e., the second classification rule).

### Fourth Embodiment

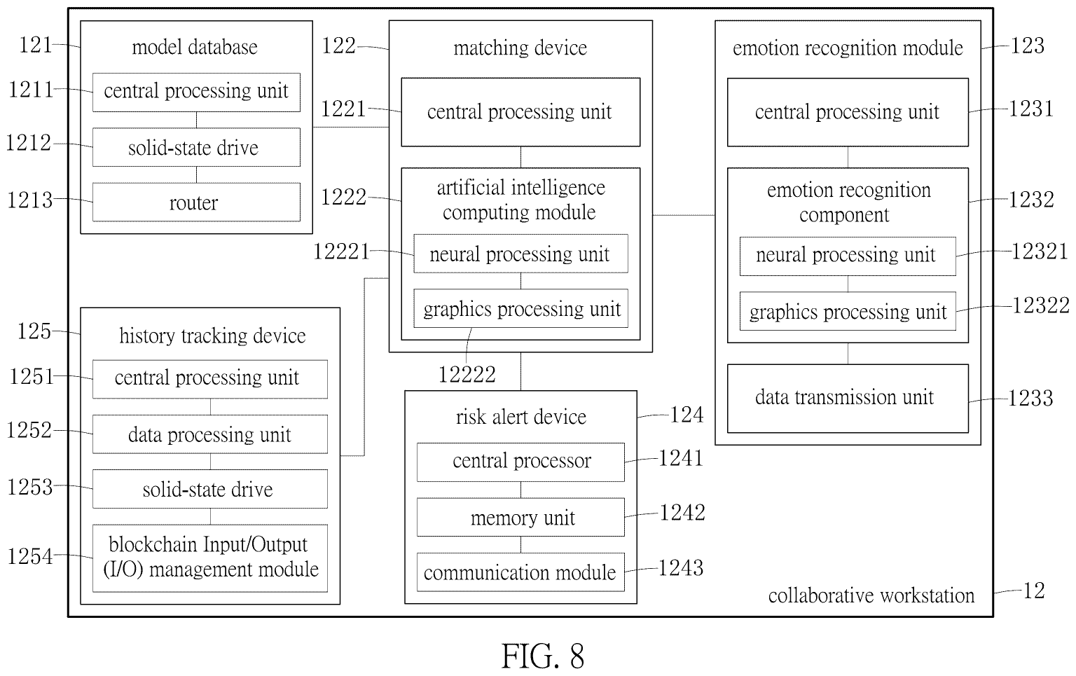

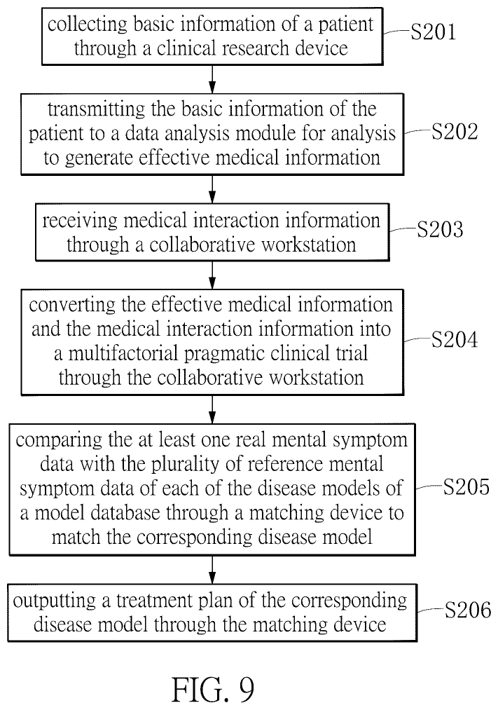

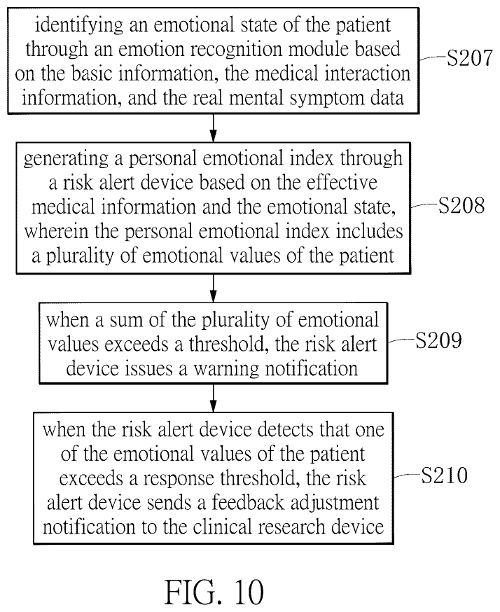

Referring to FIG. 9 and FIG. 10, a fourth embodiment of the present disclosure provides a multifactorial evidence-based analysis method, and the multifactorial evidence-based analysis method is applied to a bioinformatics platform (e.g., the AI/ML based bioinformatics platform of the first embodiment). As such, reference is also made to FIG. 1 to FIG. 4 and FIG. 8. The multifactorial evidence-based analysis method includes steps S201 to S210. It should be noted that any one of the above-mentioned steps can be omitted or adjusted according to practical requirements.

The step S201 is implemented by collecting basic information of a patient through a clinical research device.

The step S202 is implemented by transmitting the basic information of the patient to a data analysis module for analysis to generate effective medical information.

The step S203 is implemented by receiving medical interaction information through a collaborative workstation.

The step S204 is implemented by converting the effective medical information and the medical interaction information into a multifactorial pragmatic clinical trial through the collaborative workstation.

The step S205 is implemented by comparing the at least one of piece of real mental symptom data with the plurality of pieces of reference mental symptom data of each of the disease models of a model database through a matching device to match the corresponding disease model.

The step S206 is implemented by outputting a treatment plan of the corresponding disease model through the matching device.

The step S207 is implemented by identifying an emotional state of the patient through an emotion recognition module based on the basic information, the medical interaction information, and the real mental symptom data.

The step S208 is implemented by generating a personal emotional index through a risk alert device based on the effective medical information and the emotional state, wherein the personal emotional index includes a plurality of emotional values of the patient.

The step S209 occurs when a sum of the plurality of emotional values exceeds a threshold, the risk alert device issues a warning notification.

The step S210 occurs when the risk alert device detects that one of the emotional values of the patient exceeds a response threshold, the risk alert device sends a feedback adjustment notification to the clinical research device.

### BENEFICIAL EFFECTS OF THE EMBODIMENTS

Therefore, in the artificial intelligence/machine learning-based bioinformatics platform for encephalopathy and the multifactorial evidence-based analysis method provided by the present disclosure, by virtue of “the collaborative workstation translates a multifactorial pragmatic clinical trial according to the medical interaction information and the effective medical information, and the multifactorial pragmatic clinical trial includes at least one of piece of real mental symptom data” and “when the plurality of pieces of reference mental symptom data of one of the disease models matches the at least one of piece of real mental symptom data, the matching device outputs the treatment plan of the disease model that matches the at least one of piece of real mental symptom data” the element can be used to provide individualized, evidence-based treatment recommendations for patients with encephalopathy presenting complex or overlapping mental health symptoms. The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An artificial intelligence/machine learning based bioinformatics platform for encephalopathy, comprising:
an evidence-based clinical system configured to obtain real-world data of a patient, wherein the evidence-based clinical system includes:
a clinical research device capable of collecting and analyzing basic information of the patient to generate effective medical information; and
a collaborative workstation connected to the clinical research device and configured to obtain medical interaction information between a physician and the patient, wherein the collaborative workstation translates a multifactorial pragmatic clinical trial according to the medical interaction information and the effective medical information, and the multifactorial pragmatic clinical trial includes at least one of piece of real mental symptom data;
wherein the collaborative workstation includes:
a model database included a plurality of disease models, each of the disease models included a plurality of pieces of reference mental symptom data and a treatment plan, wherein, at least one of piece of the reference mental symptom data is different between any two of the disease models; and
a matching device connected to the model database, the matching device configured to compare the at least one of piece of real mental symptom data with the plurality of reference mental symptom data of each of the disease models, wherein, when the plurality of reference mental symptom data of one of the disease models matches the at least one of piece of real mental symptom data, the matching device outputs the treatment plan of the disease model that matches the at least one of piece of real mental symptom data;
wherein the real-world data includes the effective medical information and the pragmatic clinical trial; and
an evidence-based education system connected to the evidence-based clinical system and configured to selectively modifying the real-world data.

2. The artificial intelligence/machine learning based bioinformatics platform according to claim 1, wherein the collaborative workstation further includes an emotion recognition module, which is connected to the matching device, and the emotion recognition module is configured to identify an emotional state of the patient based on the basic information, the medical interaction information, and the at least one of piece of real mental symptom data.

3. The artificial intelligence/machine learning based bioinformatics platform according to claim 2, wherein the collaborative workstation further includes a risk alert device, which is connected to the matching device, the risk alert device is configured to generate a personal emotional index based on the effective medical information and the emotional state, wherein the personal emotional index includes a plurality of emotional values of the patient, and wherein, when a sum of the emotional values exceeds a threshold, the risk alert device issues a warning notification.

4. The artificial intelligence/machine learning based bioinformatics platform according to claim 1, wherein the collaborative workstation further includes a history tracking device, which is connected to the matching device, the history tracking device being configured to record and retrospectively trace patient-related the real-world data, the basic information, the effective medical information, the medical interaction information, the multifactorial pragmatic clinical trial, the real mental symptom data and the treatment plan.

5. The artificial intelligence/machine learning based bioinformatics platform according to claim 1, wherein the evidence-based clinical system is further configured to obtain multi-gene testing data of the patient; wherein the matching device is further configured to analyze the multi-gene testing data to adjust the treatment plan.

6. The artificial intelligence/machine learning based bioinformatics platform according to claim 1, wherein the evidence-based education system includes a server and a deep learning module that is electrically coupled to the server, wherein the server is used for being connected to a medical database of an official or medical institution to provide legal medical means information for the deep learning module, and the deep learning module establishes real-world evidence according to the legal medical means information, the effective medical information, and the medical interaction information, and wherein the real-world evidence is used for selectively modifying the real-world data.

7. The artificial intelligence/machine learning based bioinformatics platform according to claim 1, wherein the basic information includes sound data, and the clinical research device includes an audio collection module configured to collect and analyze the sound data, so as to generate the effective medical information of the patient.

8. The artificial intelligence/machine learning based bioinformatics platform according to claim 1, wherein the basic information includes image data, and the clinical research device includes an image collection module configured to collect and analyze the image data, so as to generate the effective medical information of the patient.

9. The artificial intelligence/machine learning based bioinformatics platform according to claim 1, wherein the basic information includes physiological data, and the clinical research device includes a physiological information collection module configured to collect and analyze the physiological data of the patient, so as to generate the effective medical information of the patient.

10. The artificial intelligence/machine learning based bioinformatics platform according to claim 1, wherein the legal medical means information includes medical history data of the patient and relevant medical regulation data.

11. A multifactorial evidence-based analysis method, which is applicable to an artificial intelligence/machine learning based bioinformatics platform for encephalopathy, comprising:
collecting basic information of a patient through a clinical research device;
transmitting the basic information of the patient to a data analysis module for analysis to generate effective medical information;
receiving medical interaction information through a collaborative workstation;
converting the effective medical information and the medical interaction information into a multifactorial pragmatic clinical trial through the collaborative workstation;
comparing the at least one of piece of real mental symptom data with the plurality of pieces of reference mental symptom data of each of the disease models of a model database through a matching device to match the corresponding disease model; and
outputting a treatment plan of the corresponding disease model through the matching device.

12. The multifactorial evidence-based analysis method according to claim 11, further comprising:
identifying an emotional state of the patient through an emotion recognition module based on the basic information, the medical interaction information, and the real mental symptom data;
generating a personal emotional index through a risk alert device based on the effective medical information and the emotional state, wherein the personal emotional index includes a plurality of emotional values of the patient; and
when a sum of the plurality of emotional values exceeds a threshold, the risk alert device issues a warning notification.

13. The multifactorial evidence-based analysis method according to claim 11, further comprising:
when the risk alert device detects that one of the emotional values of the patient exceeds a response threshold, the risk alert device sends a feedback adjustment notification to the clinical research device.

14. The multifactorial evidence-based analysis method according to claim 11, wherein the basic information includes at least one of sound data, image data, and physiological data.

15. The multifactorial evidence-based analysis method according to claim 11, further comprising:
translating a pragmatic clinical trial by use of the medical interaction information and the effective medical information, wherein the pragmatic clinical trial and the effective medical information are defined as real-world data;
obtaining legal medical means information from a medical database of an official or medical institution;
establishing real-world evidence according to the legal medical means information, the effective medical information, and the medical interaction information; and
using the real-world evidence to verify and selectively modify the real-world data.

16. The multifactorial evidence-based analysis method according to claim 15, wherein the legal medical means information includes at least one of medical history data of the patient and relevant medical regulation data.

17. The multifactorial evidence-based analysis method according to claim 16, further comprising:
collecting blood information and neuromodulation information of the patient;
categorizing a condition of the patient as a first classification rule when a glial fibrillary acidic protein in the blood information is detected to be greater than or equal to a standard value and the neuromodulation information is abnormal;
categorizing a condition of the patient as a second classification rule when only the neuromodulation information is abnormal; and
analyzing, according to the first classification rule or the second classification rule, the basic information to further obtain the effective medical information.
